(19) United States
(12) Patent Application Publication
(10) Pub. No.: US 2022/0356232 A1
(43) Pub. Date: Nov. 10, 2022

(54) A COMBINATION OF PLASMA IMMUNOGLOBULIN AND ANTIGEN-SPECIFIC IMMUNOGLOBULIN FOR THE MODIFICATION OF THE IMMUNE SYSTEM AND THE TREATMENT OR PREVENTION OF AUTOIMMUNE DISEASES

(71) Applicant: Network Immunology Inc., Vancouver (CA)

(72) Inventors: Geoffrey William Hoffmann, Vancouver, BC (CA); Reginald M. Gorczynski, Willowdale, ON (CA)

(73) Assignee: Network Immunology Inc., Vancouver, BC (CA)

(21) Appl. No.: 17/622,590

(22) PCT Filed: Jun. 26, 2020
(86) PCT No.: PCT/CA2020/050894

§ 371 Date: Dec. 23, 2021

Related U.S. Application Data
(60) Provisional application No. 62/868,146, filed on Jun. 28, 2019.

(51) Int. Cl. C07K 16/12; C07K 16/06; A61P 29/00

(52) U.S. Cl. CPC C07K 16/1282; C07K 16/06; A61P 29/00

(57) ABSTRACT

The present invention relates to the use of plasma immunoglobulin, such as intramuscular immunoglobulin, in combination with polyclonal antigen-specific immunoglobulin in the treatment or prevention of autoimmune diseases. The invention furthermore relates to a pharmaceutical composition for the treatment or prevention of autoimmune diseases comprised of plasma immunoglobulin in combination with polyclonal antigen-specific immunoglobulin.

### CROSS-REFERENCE TO RELATED APPLICATIONS

This is a U.S. national phase application under 35 U.S.C. § 371 of United States Patent Cooperation Treaty Application No. PCT/CA2020/050894, filed Jun. 26, 2020, which claims priority from U.S. Provisional Patent Application Ser. No. 62/868,146, filed in the United States Patent and Trademark Office on Jun. 28, 2019, and which incorporates by reference those PCT and Provisional applications in their entireties.

### FIELD OF THE INVENTION

This invention relates to the prevention or treatment of autoimmune diseases. In particular, this invention relates to the use of pooled immunoglobulin in combination with antigen-specific polyclonal immunoglobulin in the prevention or treatment of autoimmune diseases.

### BACKGROUND OF THE INVENTION

Autoimmune diseases afflict a significant fraction of the human population and of the population of pets.

Inflammatory bowel disease (IBD) is an autoimmune-like inflammatory disorder of the gastrointestinal tract with major clinical entities, Crohn's disease (CD) and ulcerative colitis (UC) [1,2]. While the etiology of IBD is unclear, both genetic susceptibility and intestinal microbial and environmental factors likely contribute to perturbation of the mucosal immune system, with overproduction of inflammatory cytokines, infiltration of cells of myeloid and lymphoid origin, and resultant disease [3,4]. Amongst the inflammatory cytokines implicated in the pathogenesis of IBD are IFNγ, IL-1β, IL-6, IL-17 and TNFα, [1,5,6].

WO 2017/024404 A1 discloses that the combination of antigen-specific antibodies and antiidiotypic antibodies may be used to treat or attenuate: (1) metastasis in an inflammatory breast cancer model in mice; and (2) autoimmune disease using inflammatory colitis induced in mice as an example.

It has also been demonstrated that the combination of antigen-specific antibodies and antiidiotypic antibodies from the same species of mouse may be used to treat or prevent allergic diseases using mice induced with ovalbumin as an example [7].

Additionally, in a DSS-induced inflammatory colitis model in mice, attenuation of disease has been seen if mice received weekly injections (×5) of a combination of polyclonal immune Ig and polyclonal anti-idiotype Ig, beginning before DSS exposure [7]. Dextran sodium sulfate (DSS) is a commonly used chemical for induction of murine colitis [8] that has many similarities to human IBD. It has also bee shown, using this same combined Ig treatment approach to modify allergic-type responses in BALB/c mice to ovalbumin (OVA), that at least once mechanism of action of this therapy involved induction and/or expansion of Treg populations through the perturbation of immunological networks [9, 10].

It is an object of this invention to provide an improved pharmaceutical compositions and methods for the treatment or prevention of autoimmune diseases.

This and other objects of the invention will be better understood by reference to the detailed description of the preferred embodiment which follows. Note that the object referred to above is a statement of what motivated the invention rather than promises. Not all the objects are necessarily met by all embodiments of the invention described below or by the invention defined by each of the claims.

### SUMMARY OF THE INVENTION

According to one aspect of the invention, a combination of plasma immunoglobulin and antigen-specific immunoglobulin may be used for the treatment or prevention of autoimmune disease. In a further aspect, the autoimmune disease involves inflammation. The plasma immunoglobulin may be homologous, and the antigen-specific immunoglobulin may be homologous. Alternatively, the plasma immunoglobulin may be heterologous, and the antigen-specific immunoglobulin may be heterologous. In a further aspect, the plasma immunoglobulin is pooled plasma immunoglobulin and the antigen-specific immunoglobulin is anti-Tetanus toxoid Ig.

According to a further aspect, the invention is a pharmaceutical composition for the treatment or prevention of autoimmune disease. The pharmaceutical composition comprises plasma immunoglobulin and antigen-specific immunoglobulin. In a further aspect, the autoimmune disease involves inflammation. The plasma immunoglobulin may be homologous, and the antigen-specific immunoglobulin may be homologous. Alternatively, the plasma immunoglobulin may be heterologous, and the antigen-specific immunoglobulin may be heterologous. In a further aspect, the plasma immunoglobulin is pooled plasma immunoglobulin and the antigen-specific immunoglobulin is anti-Tetanus toxoid Ig.

According to a further aspect, the invention is a method of treating or preventing autoimmune disease comprising administering injections of plasma immunoglobulin and antigen-specific immunoglobulin. In a further aspect, the autoimmune disease involves inflammation. The plasma immunoglobulin may be homologous, and the antigen-specific immunoglobulin may be homologous. Alternatively, the plasma immunoglobulin may be heterologous, and the antigen-specific immunoglobulin may be heterologous. In a further aspect, the plasma immunoglobulin is pooled plasma immunoglobulin and the antigen-specific immunoglobulin is anti-Tetanus toxoid Ig.

The foregoing may cover only some of the aspects of the invention. Other aspects of the invention may be appreciated by reference to the following description of at least one preferred mode for carrying out the invention in terms of one or more examples. The following mode(s) for carrying out the invention is not a definition of the invention itself, but is only an example that embodies the inventive features of the invention.

### BRIEF DESCRIPTION OF THE FIGURES

At least one mode of carrying out the invention in terms of several examples will be described by reference to the drawings below in which:

FIG. 1A is a graph showing the attenuation of an anti-OVA IgE response by combined injections of IMIG and polyclonal anti-Tetanus toxoid Ig.

FIG. 1B is a graph showing no change in an anti-OVA IgG response by combined injections of IMIG and polyclonal anti-Tetanus toxoid Ig.

FIG. 1C is a graph showing OVA-induced IL-4 in splenocytes of OVA immunized mice receiving combined injections of IMIG and polyclonal anti-Tet Ig.

FIG. 1D is a graph showing OVA-induced IL-2 in splenocytes of OVA immunized mice receiving combined injections of IMIG and polyclonal anti-Tet Ig.

FIG. 2A is a graph showing OVA-induced IL-2 in splenocytes of OVA immunized mice injected with IMIG and anti-Varicella Ig.

FIG. 2B is a graph showing OVA-induced IL-4 in splenocytes of OVA immunized mice injected with IMIG and anti-Varicella Ig.

FIG. 3A is a graph showing the total serum IgG levels in dog blood before sensitization with peanut butter and treatment with dog IMIG and dog anti-Rabies immune Ig or treatment with human IMIG and human anti-Tetanus Ig.

FIG. 3B is a graph showing the total serum IgG levels in dog blood after sensitization with peanut butter and treatment with dog IMIG and dog anti-Rabies immune Ig or treatment with human IMIG and human anti-Tetanus Ig.

FIG. 3C is a graph showing the total serum IgE levels in dog blood before sensitization with peanut butter and treatment with dog IMIG and dog anti-Rabies immune Ig or treatment with human IMIG and human anti-Tetanus Ig.

FIG. 3D is a graph showing the total serum IgE levels in dog blood after sensitization with peanut butter and treatment with dog IMIG and dog anti-Rabies immune Ig or treatment with human IMIG and human anti-Tetanus Ig.

FIG. 3E is a graph showing IL-2 production by ConA-stimulated peripheral blood lymphocytes of dogs before sensitization with peanut butter and treatment with dog IMIG and dog anti-Rabies immune Ig or treatment with human IMIG and human anti-Tetanus Ig.

FIG. 3F is a graph showing IL-2 production by ConA-stimulated peripheral blood lymphocytes of dogs after sensitization with peanut butter and treatment with dog IMIG and dog anti-Rabies immune Ig or treatment with human IMIG and human anti-Tetanus Ig.

FIG. 3G is a graph showing IL-4 production by ConA-stimulated peripheral blood lymphocytes of dogs before sensitization with peanut butter and treatment with dog IMIG and dog anti-Rabies immune Ig or treatment with human IMIG and human anti-Tetanus Ig.

FIG. 3H is a graph showing IL-4 production by ConA-stimulated peripheral blood lymphocytes of dogs after sensitization with peanut butter and treatment with dog IMIG and dog anti-Rabies immune Ig or treatment with human IMIG and human anti-Tetanus Ig.

FIG. 3I is a graph showing the ratio of IL-2/IL-4 production levels by ConA-stimulated peripheral blood lymphocytes of dogs before and after sensitization with peanut butter and treatment with dog IMIG and dog anti-Rabies immune Ig or treatment with human IMIG and human anti-Tetanus Ig.

FIG. 3J is a graph showing the IL-2 and IL-4 production by peanut butter Ag-stimulated peripheral blood lymphocytes of dogs after sensitization with peanut butter and treatment with dog IMIG and dog anti-Rabies immune Ig or treatment with human IMIG and human anti-Tetanus Ig.

FIG. 3K is a graph showing the ratio of IL-2/IL-4 production levels by peanut butter Ag-stimulated peripheral blood lymphocytes of dogs after sensitization with peanut butter and treatment with dog IMIG and dog anti-Rabies immune Ig or treatment with human IMIG and human anti-Tetanus Ig.

FIG. 4A is a graph showing OVA-specific IgE serum levels in OVA immunized mice after combined infusion of varying doses of IMIG and anti-Tetanus Ig.

FIG. 4B is a graph showing OVA-specific IgG serum levels in OVA immunized mice after combined infusion of varying doses of IMIG and anti-Tetanus Ig.

FIG. 4C is a graph showing IL-4 production by OVA-stimulated splenocytes in OVA immunized mice after combined infusion of varying doses of IMIG and anti-Tetanus Ig.

FIG. 4D is a graph showing IL-2 production by OVA-stimulated splenocytes in OVA immunized mice after combined infusion of varying doses of IMIG and anti-Tetanus Ig.

FIG. 4E is a graph showing the ratio of IL-2/IL-4 production levels by OVA-stimulated splenocytes of OVA immunized mice after combined infusion of varying doses of IMIG and anti-Tetanus Ig.

FIG. 5A is a graph showing OVA-specific IgE serum levels in mice sensitized to OVA along with repeated injections of human IMIG and/or human anti-Tetanus Ig.

FIG. 5B is a graph showing OVA-specific IgG serum levels in mice sensitized to OVA along with repeated injections of human IMIG and/or human anti-Tetanus Ig.

FIG. 5C is a graph showing OVA-induced IL-4 production in cultured splenocytes of mice sensitized to OVA along with repeated injections of human IMIG and/or human anti-Tetanus Ig.

FIG. 5D is a graph showing OVA-induced IL-2 production in cultured splenocytes of mice sensitized to OVA along with repeated injections of human IMIG and/or human anti-Tetanus Ig.

FIG. 5E is a graph showing OVA-induced IL-33 production in cultured splenocytes of mice sensitized to OVA along with repeated injections of human IMIG and/or human anti-Tetanus Ig.

FIG. 5F is a graph showing OVA-induced IL-31 production in cultured splenocytes of mice sensitized to OVA along with repeated injections of human IMIG and/or human anti-Tetanus Ig.

FIG. 6 is a schematic showing the induction of chronic colitis in C57BL/6 mice, with three cycles of DSS exposure, the timing of intramuscular injections of immune Ig and anti-idiotype Ig to attenuate disease, and the timing of injection of anti-CD4mAb in studies used to explore the role of such cells in disease onset and suppression of disease.

FIG. 7A is a graph showing the protection from weight loss in mice with chronic colitis receiving immune Ig and anti-idiotype Ig or Solu-Cortef.

FIG. 7B is a graph showing protection from shortened colon length in mice with chronic colitis receiving immune Ig and anti-idiotype Ig or Solu-Cortef.

FIG. 8 is a graph showing protection from inflammatory cytokine release from explants of mice with chronic colitis receiving immune Ig and anti-idiotype Ig or Solu-Cortef.

FIG. 9A is a graph showing weight loss in mice with DSS-chronic colitis receiving in addition either Solu-Cortef or combined Anti-TetIg and IMIG.

FIG. 9B is a graph showing the effect of additional anti-CD4 mAb treatment on weight loss in mice with DSS-chronic colitis receiving in addition either Solu-Cortef or combined Anti-TetIg and IMIG.

FIG. 10 is a graph showing the effect of anti-CD4 mAb treatment on cytokine production in colon explant cultures from mice with DSS-chronic colitis receiving in addition either Solu-Cortef or combined Anti-TetIg and IMIG.

FIG. 11 is a schematic showing the protocol for DSS treatment, and sequence of delivery of immune Ig and anti-idiotype Ig (given intramuscularly in separate locations), to C57BL/6 mice to assess attenuation of inflammatory colitis.

FIG. 12A is a graph showing the changes in body weight in mice with DSS-induced acute colitis receiving either Solu-Cortef or combined immune Ig and anti-idiotype Ig as in FIG. 11.

FIG. 12B is a graph showing the changes in colon length in mice with DSS-induced acute colitis receiving either Solu-Cortef or combined immune Ig and anti-idiotype Ig as in FIG. 11.

FIG. 12C is a graph showing the changes in cytokine production from colonic explants in mice with DSS-induced acute colitis receiving either Solu-Cortef or combined immune Ig and anti-idiotype Ig as in FIG. 11.

FIG. 13A shows representative hematoxylin and eosin (H&E) staining of a colon segment from control (no DSS) mice.

FIG. 13B shows representative H&E staining of a colon segment from DSS only treated mice.

FIG. 13C shows representative H&E staining of a colon segment from DSS treated mice receiving Solu-Cortef.

FIG. 13D shows representative H&E staining of a colon segment from DSS treated mice receiving IMIG+Anti-TetIg.

FIG. 14A is a graph showing the protection from weight loss in mice with chronic colitis (see FIG. 6) receiving further immunomodulation with Solu-Cortef or immune Ig and anti-idiotype Ig, but with IMIG and anti-Tet Ig given only after 2 complete cycles of DSS (i.e. commencing at day 21, not day 0, and repeated at days 28 & 35).

FIG. 14B is a graph showing shortened colon length in mice with chronic colitis (see FIG. 6) receiving further immunomodulation with Solu-Cortef or immune Ig and anti-idiotype Ig, but with IMIG and anti-Tet Ig given only after 2 complete cycles of DSS (i.e. commencing at day 21, not day 0, and repeated at days 28 & 35).

### DETAILED DESCRIPTION OF AT LEAST ONE MODE FOR CARRYING OUT THE INVENTION IN TERMS OF EXAMPLE(S)

All publications, patent applications, patents, and other references mentioned herein are incorporated by reference in their entirety.

The pharmaceutical compositions of the present invention are a combination of pooled plasma immunoglobulin and antigen-specific polyclonal immunoglobulin.

Plasma immunoglobulin is typically prepared from the serum of at least 1000 donors and has been used to treat patients with primary immunodeficiency diseases. Depending on how the plasma immunoglobulin is formulated and administered, it may be referred to as intramuscular immunoglobulin (“IMIG”), intravenous immunoglobulin (“IVIG”), subcutaneous immunoglobulin (“SCIG”), or intraperitoneal immunoglobulin (“IPIG”).

Antigen-specific polyclonal immunoglobulin is immunoglobulin prepared with a high antibody count against a specific pathogen, such as Varicella-zoster virus, or against a specific antigen, such as Tetanus toxoid.

As will be described in detail below, the inventors conducted experiments using various antigen-specific immunoglobulins against various antigens. In view of the significant differences in preparation and chemical composition between the antigen-specific immunoglobulins used (polyclonal anti-Tetanus immunoglobulin, polyclonal anti-Rabies immunoglobulin, and polyclonal anti-Varicella immunoglobulin), the combination of plasma immunoglobulin with any antigen-specific immunoglobulin (including but not limited to polyclonal anti-Rh immunoglobulin and polyclonal anti-hepatitis B immunoglobulin) is predicted to result in a similar modification of the immune system of the treated human or animal and to similarly prevent and/or treat allergic diseases.

For the treatment and prevention of allergic diseases in humans and pets, the plasma immunoglobulin component of the combination may be plasma immunoglobulin approved for use in humans such as, but not limited to, Gamunex™ and Hizentra™.

The plasma immunoglobulin and antigen-specific polyclonal immunoglobulin may be administered in any suitable manner. For example, the antibodies may be administered in a non-immunogenic form, that is without an adjuvant, in a non-immunogenic amount, for example intramuscularly, intravenously, subcutaneously, or intraperitoneally. A pharmaceutical composition comprising the combination of plasma immunoglobulin and antigen-specific polyclonal immunoglobulin may include a pharmaceutically acceptable carrier, such as buffered saline, phosphate buffered saline, or phosphate buffered saline at neutral pH.

Another aspect of the invention is a kit comprising aliquots of plasma immunoglobulin, aliquots of polyclonal antigen-specific immunoglobulin, and instructions directing use of such two different aliquots for the treatment or prevention of allergic disease.

Another aspect of the invention is a kit comprising aliquots of a mixture of plasma immunoglobulin and polyclonal antigen-specific immunoglobulin and instructions directing use of such aliquots for the treatment or prevention of allergic disease.

The antibodies may be administered at any suitable site and time. However, the antibodies are preferably administered contemporaneously or substantially contemporaneously. The antibodies may be administered in separate compositions sequentially or contemporaneously or together as a mixture.

A. The Treatment and Prevention of Allergic Diseases

### Example 1

Allergy Experiment Protocol with Polyclonal Human IMIG and Polyclonal Human Anti-Tetanus Ig

Five BALB/c mice (8 weeks of age at initiation of study) were used per group. All mice except a control group (Group 1—see below) received ovalbumin (10 μg OVA plus Al(OH)₃) intraperitoneally in 0.3 ml phosphate buffered saline (“PBS”) on day 0 and day 14 with a further boost on day 56. All mice received egg white solution (filtered 20% (w/v) EWS) in their drinking water from day 14.

Mice receiving intramuscular immunoglobulin (Gamunex™ 25 μg: Grifols) were given intramuscular injections in 0.05 ml PBS in the left gluteus muscle at days −2, 7, 14, 21, 28, 35, and 42. Animals receiving Anti-Tetanus immune Ig (HyperTET™ 25 μg: Grifols) were given intramuscular injections in 0.05 ml PBS in the right deltoid muscle at days −2, 7, 14, 21, 28, 35, and 42. In some cases IMIG or Anti-Tetanus was absorbed with Tetanus toxoid (×3) before use. The control groups received PBS only in the same sites.

The following groups were used.
Group 1: OVA⁻/EWS⁺
Group 2: OVA⁻/EWS⁺
Group 3: As for group 2+IMIG
Group 4: As for group 2+Anti-Tet Ig
Group 5: As for group 2+IMIG+anti-Tet Ig
Group 6: As for group 2+IMIG (absorbed×3 with Tet)+ant-Tet Ig
Group 7: As for group 2+IMIG+anti-Tet Ig (absorbed with Tet×3)

All mice were sacrificed at day 63 of the study. On sacrifice, serum IgE to OVA obtained by cardiac puncture was measured by ELISA using plates coated with 100 ng/well of OVA and developed with HRP-anti-mouse IgE and appropriate substrate.

In addition, 5×10⁶ splenocytes from individual animals were challenged in vitro in 2 ml medium with 1 μg/ml OVA for 72 hr and IL-2/IL-4 measured in culture supernatants using commercial ELISA Kits (eBIOSciences).

Results

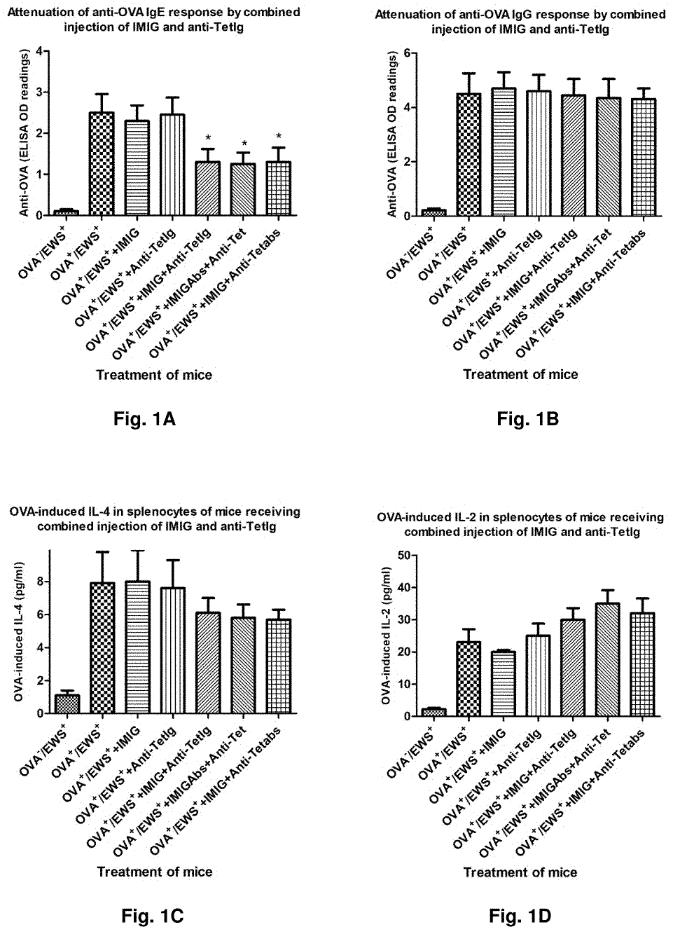

FIG. 1A shows that a combination of IMIG and polyclonal anti-Tetanus Ig results in significant suppression of OVA-specific IgE and FIG. 1B shows no significant effect on OVA-specific IgG. Referring now to FIG. 1A, absorption of IMIG or polyclonal anti-Tetanus with Tetanus (groups 6 and 7) made no difference. This suggests that the active pharmaceutical ingredient is not the anti-Tetanus antibodies.

FIG. 1C shows a decrease in the levels of OVA-induced IL-4 in the splenocytes of mice in groups 5, 6, and 7 that received combined injections of IMIG and anti-Tetanus Ig. When considered together with FIG. 1D showing OVA-induced IL-2 levels, it is apparent that the ratio of IL-4 to IL-2 is smaller for the groups treated with IMIG plus anti-Tetanus Ig (groups 5, 6, and 7) than the control groups 2, 3 and 4. This result is consistent with suppression of allergy and the results shown in FIGS. 1A and 1B.

### Example 2

Allergy Experiment Protocol with Polyclonal Human IMIG and Polyclonal Human Anti-Varicella Ig

Eight BALB/c mice (8 weeks of age at initiation of study) were used per group. All mice except a control group (Group 1—see below) received ovalbumin (10 μg OVA plus Al(OH)₃) intraperitoneally in 0.3 ml PBS on day 0, day 14, and day 42. All mice received egg white solution (filtered 20% (w/v) EWS) in their drinking water from days 14-42.

IMIG (Gamunex™ 25 μg: Grifols) and anti-Varicella Ig (25 μg: Grifols) was infused weekly intravenously from days 7-35.

The following groups were used.
Group 1: OVA⁻/EWS⁺
Group 2: OVA⁻/EWS⁺
Group 3: As for group 2+IMIG
Group 4: As for group 2+Anti-Varicella Ig
Group 5: As for group 2+IMIG+anti-Varicella Ig

All mice were sacrificed at day 49 of the study. On sacrifice, serum IgE to OVA obtained by cardiac puncture was measured by ELISA using plates coated with 100 ng/well of OVA and developed with HRP-anti-mouse IgE and appropriate substrate.

In addition, 5×10⁶ splenocytes from individual animals were challenged in vitro in 2 ml medium with 1 μg/ml OVA for 72 hr and IL-2/IL-4 measured in culture supernatants using commercial ELISA Kits (eBIOSciences).

Results

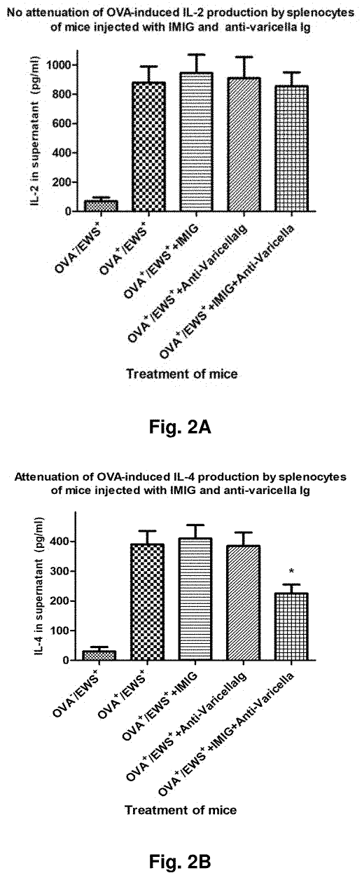

FIG. 2A shows that there was no attenuation of IL-2 production from the OVA-stimulated individual splenocytes of mice. In contrast, FIG. 2B shows a clear attenuation of IL-4 production. These results are consistent with the study done in EXAMPLE 1 using combined injection of IMIG and anti-Tetanus Ig (see FIGS. 1C and 1D).

### Example 3

Allergy experiment protocol with polyclonal Human IMIG and polyclonal human Anti-Tetanus Ig, or polyclonal Dog Ig and dog anti-Rabies Ig, using Beagle dogs sensitized to peanut butter

In a modification of the protocol in EXAMPLE 1 and EXAMPLE 2, the inventors considered whether they could attenuate allergic sensitization in large animals (Beagle dogs) where previous literature reports indicate a high prevalence (>80%) of induced allergic sensitization to peanut butter applied topically.

Animals received weekly topical exposure to peanut butter (abdomen) with the first exposure occurring 1 week before treatment. The next 5 exposures of peanut better were with 5 weekly treatments with combined dog Igs (IMIG and pooled dog anti-Rabies immune Ig) or with combined human Igs (IMIG and Anti-Tet Ig). After 5 weekly treatments all animals received a further 3 treatments given at 14 d intervals of the same Ig mixes. Finally, all dogs received an oral challenge with peanut butter, and serum IgG, serum IgE, and peanut butter induced IL-2/IL-4 production was measured.

Dogs receiving dog intramuscular immunoglobulin (Innovative Research, USA) were given 1 mg/kg intramuscular injections in 0.5 ml PBS in the gluteus muscle and dogs receiving pooled dog anti-Rabies immune Ig (prepared from pooled dogs re-immunized with rabies vaccine) were given 1 mg/kg intramuscular injections in 0.5 ml PBS in the opposite gluteus muscle.

Dogs receiving human intramuscular immunoglobulin (Gamunex™: Grifols) were given 1 mg/kg intramuscular injections in 0.5 ml PBS in the gluteus muscle and dogs receiving human Anti-Tetanus Ig (HyperTET™: Grifols) were given 1 mg/kg intramuscular injections in 0.5 ml PBS in the opposite gluteus muscle.

Results

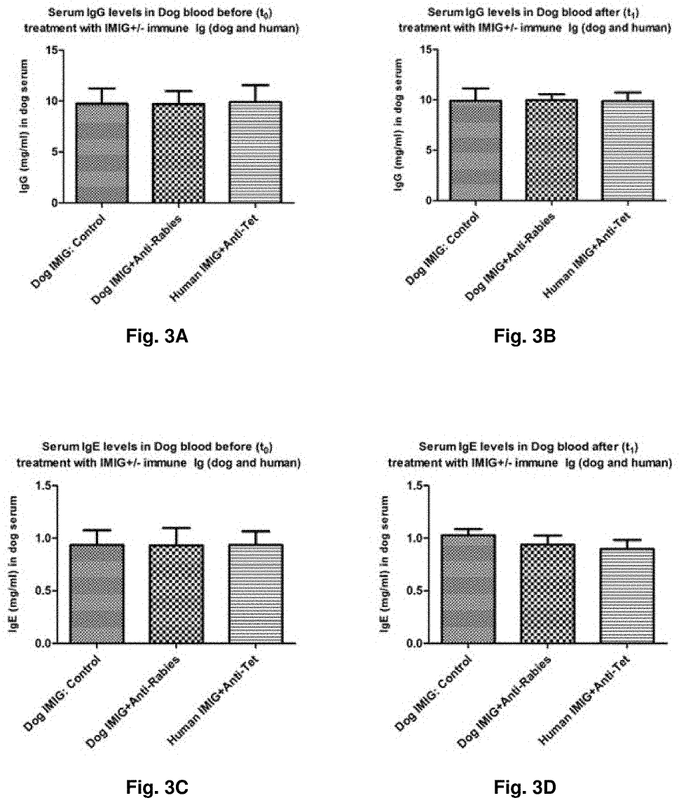

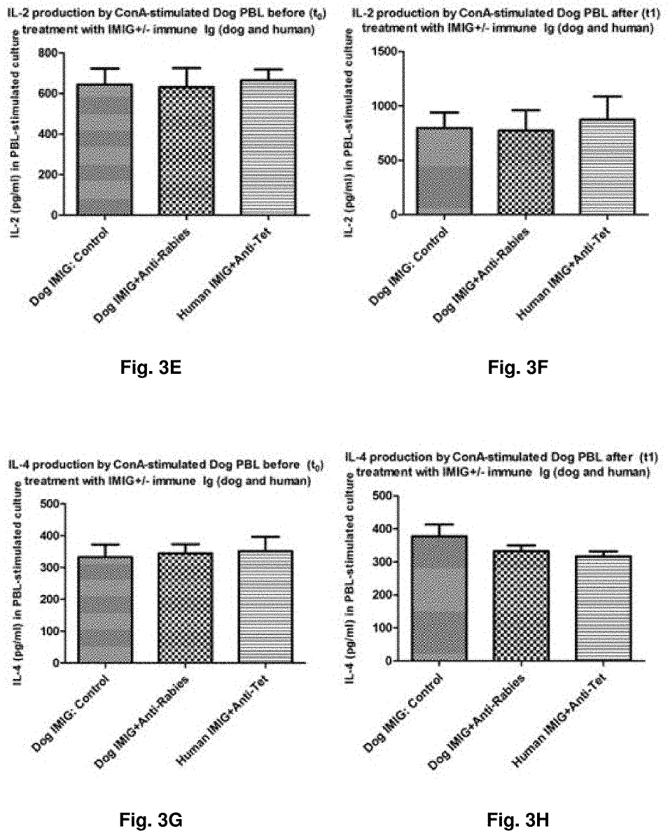

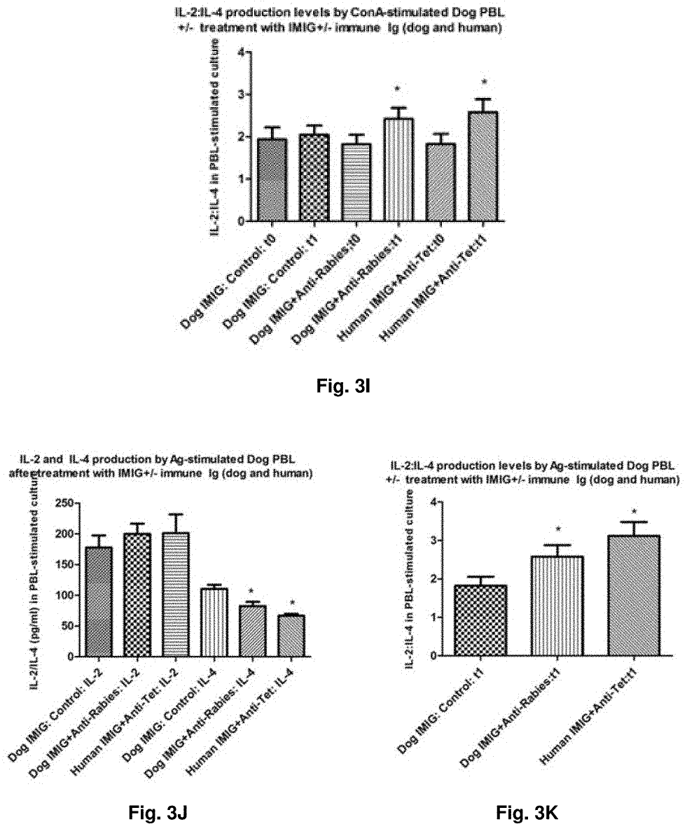

FIGS. 3A and 3B show a comparison of the total serum IgG levels at the start (t0) and end (t1) of the study respectively. FIGS. 3C and 3D show a comparison of the total serum IgE levels at the start (t0) and end (t1) of the study respectively. While there were no significant changes in the IgG levels, a reduction in the IgE levels was observed both for the treatment with dog IMIG and dog anti-Rabies Ig and for the treatment with human IMIG and human anti-Tetanus Ig.

FIGS. 3E and 3F show a comparison of the IL-2 production by conconavalin A (ConA) in dog peripheral blood lymphocytes (PBL) before (t0) and after (t1) treatment respectively.

FIGS. 3G and 3H shows a comparison of the IL-4 production by Con-A in dog PBL before (t0) and after (t1) treatment respectively.

FIG. 3I shows a comparison of the IL-2/IL-4 induction by ConA in dog PBL before/after Ig treatment. Note that even for this polyclonal response, the IL-2/IL-4 ratio is enhanced in dogs after treatment with either dog IgGs or human IgGs (*, p<0.05).

FIG. 3J shows that IL-2 and IL-4 productions by peanut butter stimulated dog PBL after treatment with dog IMIG and dog anti-Rabies Ig and after treatment with human IMIG and human anti-Tetanus Ig. FIG. 3K shows the IL-2:IL-4 ratios after treatment. Consistent with the other experiments, IL-4 production was attenuated by Ig treatment, but IL-2 production was not. Additionally, the IL-2:IL-4 ratios were elevated after treatment (*, p<0.05).

### Example 4

Dose Response Study—Polyclonal Human IMIG and Polyclonal Human Anti-Tetanus Ig in Pre-Immune Mice

The inventors also conducted a dose response study using polyclonal human IMIG and polyclonal human anti-Tetanus Ig in pre-immune mice.

In the dose response study, all groups had five BALB/c mice each. Mice were approximately 25 grams. All mice received OVA (10 μg OVA plus Al(OH)₃) on day 0 and day 14 with a boost on day 56. All mice received EWS (filtered 20% (w/v) egg white solution in their drinking bottle) from day 14.

Mice began treatment with immunoglobulin injections on days 56, 63, 70, 77, and 84.

The following groups were used:
Group 1 (control): OVA immunization and EWS
Group 2: As for Group 1+IMIG (250 μg/mouse)+anti-Tet Ig (10 μg/mouse)
Group 3: As for Group 1+IMIG (250 μg/mouse)+anti-Tet Ig (50 μg/mouse)
Group 4: As for Group 1+IMIG (250 μg/mouse)+anti-Tet Ig (250 μg/mouse)
Group 5: As for Group 1+IMIG (50 μg/mouse)+anti-Tet Ig (10 μg/mouse)
Group 6: As for Group 1+IMIG (50 μg/mouse)+anti-Tet Ig (50 μg/mouse)
Group 7: As for Group 1+IMIG (50 μg/mouse)+anti-Tet Ig (250 μg/mouse)
Group 8: As for Group 1+IMIG (10 μg/mouse)+anti-Tet Ig (10 μg/mouse)
Group 9: As for Group 1+IMIG (10 μg/mouse)+anti-Tet Ig (50 μg/mouse)
Group 10: As for Group 1+IMIG (10 μg/mouse)+anti-Tet Ig (250 μg/mouse)
Group 11: As for Group 1+IMIG (50 μg/mouse)+anti-Tet Ig (50 μg/mouse)+CoQ10 ip (100 μg/mouse) q2d from days 56-90
Group 12: As for Group 1+IMIG (250 μg/mouse)
Group 13: As for Group 1+Anti-Tet Ig (50 μg/mouse)

A final boost with OVA was on day 90 and the mice were sacrificed on day 97.

Serum OVA-specific IgG/IgE was measured by ELISA. Additionally, 2×10⁶ splenocytes were challenged in vitro in 2 ml medium with 1 μg/ml OVA for 72 hr and IL-2/IL-4 was measured in culture supernatants using commercial ELISA Kits (BioLegend™)

Results

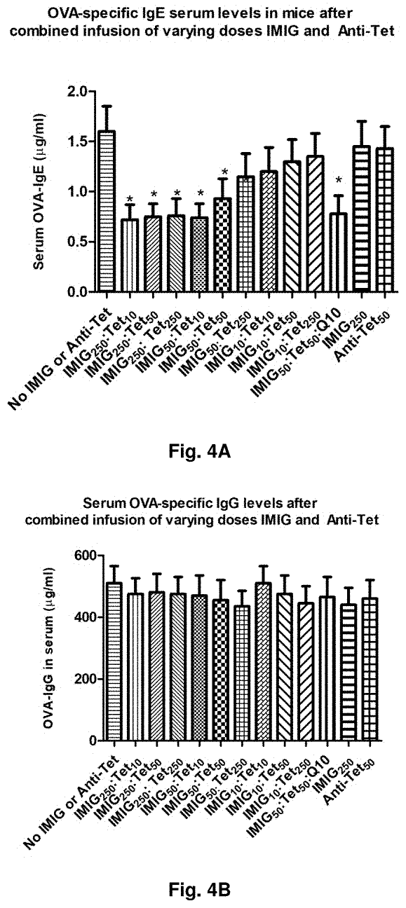

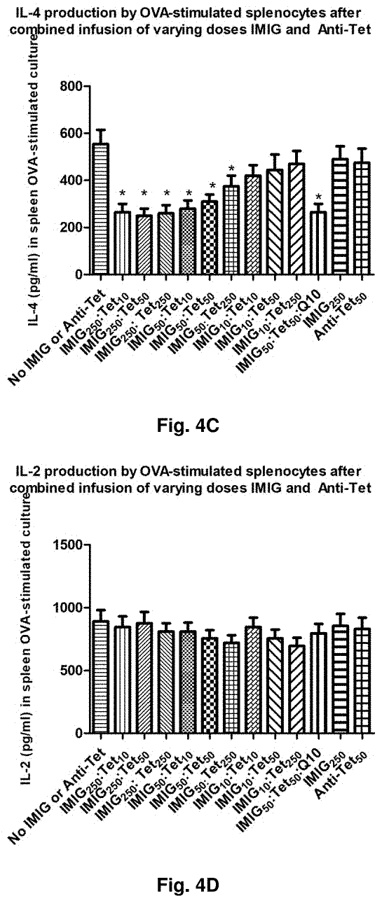

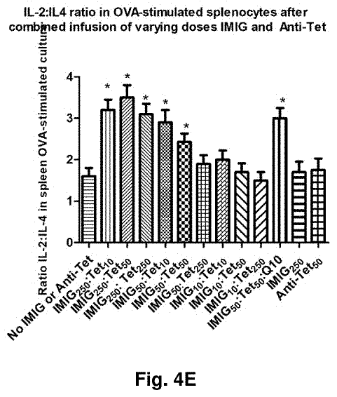

FIGS. 4A and 4B show that optimal suppression of IgE responses occurred with IMIG doses of 250 and 50 μg/mouse and across a broad spectrum of anti-Tet dosing (from 10-250 μg/mouse). At lower IMIG doses (10 μg/mouse) suppression was less evident. There was some synergy in suppression at lower doses of IMIG (50) and anti-Tet (50) if animals also received CoQ10 as anti-oxidant every 2 d (100 μg/mouse). No attenuation of IgG responses occurred (FIG. 4B).

FIGS. 4C, 4D, and 4E confirm the data of FIGS. 4A and 4B using IL-4 and IL-2 production as readout. Again suppression of IL-4 but not IL-2 production was seen with IMIG doses of 250 and 50 μg/mouse and across a broad spectrum of anti-Tet dosing (from 10-250 μg/mouse). At lower IMIG doses (10 μg/mouse) suppression was less evident. There was synergy in suppression at lower doses of IMIG (50) and anti-Tet (50) if animals also received CoQ10 as anti-oxidant every 2 d (100 μg/mouse). These data are further emphasized by comparison of IL-2:IL-4 ratios as shown in FIG. 4E.

### Example 5

Dose Response Study—Polyclonal Human IMIG and Polyclonal Human Anti-Tetanus Ig in Pre-Immune Mice

A further dose response study using 50 μg IMIG per mouse and varying doses of anti-Tetanus immunoglobulin was conducted.

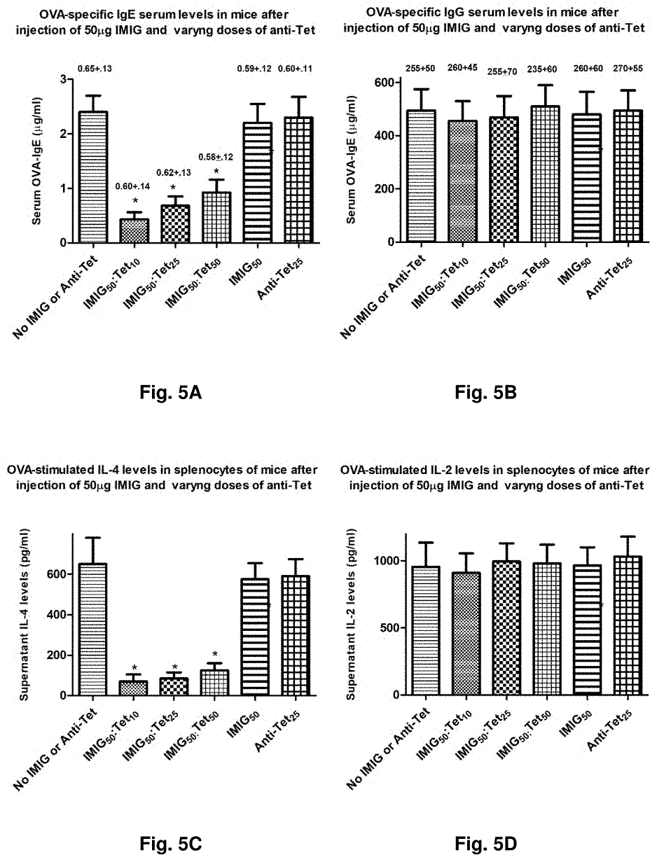

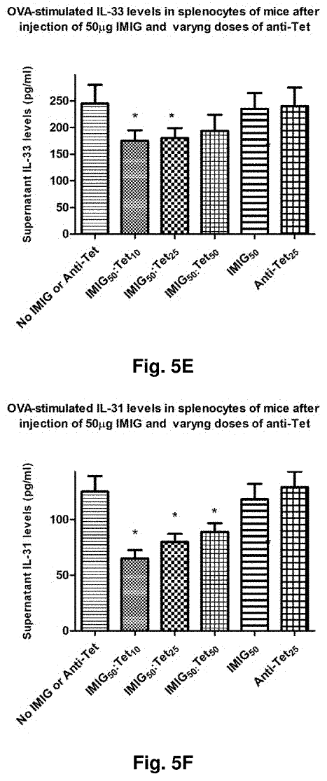

FIGS. 5A and 5B show the attenuation of OVA IgE but not IgG responses in BALB/c mice sensitized to OVA along with repeated (×5) weekly injections of human IMIG and/or human anti-Tetanus immunoglobulin.

FIGS. 5C and 5D show the attenuation of OVA-induced IL-4 but not IL-2 responses from culture splenocytes of BALB/c mice sensitized to OVA along with repeated (×5) weekly injections of human IMIG and/or human anti-Tetanus immunoglobulin.

FIGS. 5E and 5F show the attenuation of OVA-induced IL-33 and IL-31 responses from cultured splenocytes of BALB/c mice sensitized to OVA along with repeated (×5) weekly injections of human IMIG and/or human anti-Tetanus immunoglobulin.

This dosage study shows that it is possible to attenuate antigen-specific IgE production in a first species (BALB/c mice), in concert with the release of the IL-4/IL-31/IL-33 cytokines implicated in the development of allergic responses, by giving the first species (BALB/c mice) a series (5× weekly) injections of pooled plasma immunoglobulin (IMIG) from a second species (human) and pooled antigen specific immunoglobulin (anti-Tetanus Ig) from the second species (human). Accordingly, a first species may be treated using pooled plasma immunoglobulins and antigen specific immunoglobulins derived from a different species, likely due to the evolutionary conservation between immunoglobulins in vertebrates.

B. The Treatment and Prevention of Autoimmune Diseases

EXAMPLES 1-5 above show that the combination of plasma immunoglobulin and antigen specific immunoglobulin can be used to prevent or treat allergic diseases. Since the treatment or prevention of allergic disease using a combination of antiidiotypic immunoglobulin and antigen-specific immunoglobulin is known, it is believed that plasma immunoglobulin and antiidiotypic immunoglobulin contain the same active ingredient(s).

Additionally, as the combination of antiidiotypic immunoglobulin and antigen-specific immunoglobulin has been shown in WO 2017/024404 A1 to treat or prevent inflammation and autoimmune disease, it is predicted that the combination of plasma immunoglobulin and antigen-specific immunoglobulin will also treat or prevent inflammation and autoimmune diseases in humans and pets.

The combination of plasma immunoglobulin and antigen-specific immunoglobulin may be particularly effective in the treatment or prevention of autoimmune diseases where the attenuation of inflammation is important, such as inflammatory colitis.

As with the allergic disease experiments, it is also predicted the combination of plasma immunoglobulin with any antigen-specific immunoglobulin (including but not limited to polyclonal anti-Rh immunoglobulin and polyclonal anti-hepatitis B immunoglobulin) will prevent and/or treat various autoimmune diseases.

The plasma immunoglobulin component of the combination may be plasma immunoglobulin approved for use in humans such as, but not limited to, Gamunex™ and Hizentra™.

The plasma immunoglobulin and antigen-specific polyclonal immunoglobulin may be administered in any suitable manner. For example, the antibodies may be administered in a non-immunogenic form, that is without an adjuvant, in a non-immunogenic amount, for example intramuscularly, intravenously, subcutaneously, or intraperitoneally. A pharmaceutical composition comprising the combination of plasma immunoglobulin and antigen-specific polyclonal immunoglobulin may include a pharmaceutically acceptable carrier, such as buffered saline, phosphate buffered saline, or phosphate buffered saline at neutral pH.

Another aspect of the invention is a kit comprising aliquots of plasma immunoglobulin and aliquots of polyclonal antigen-specific immunoglobulin and instructions for use.

The antibodies may be administered at any suitable site and time. However, the antibodies are preferably administered contemporaneously or substantially contemporaneously. The antibodies may be administered in separate compositions sequentially or contemporaneously or together as a mixture.

The inventors have conducted additional experiments to verify the prediction that the combination pooled plasma immunoglobulin and antigen-specific immunoglobulin can be used to prevent or treat other diseases beyond allergic diseases. In particular, the inventors have now investigated attenuation of an inflammatory colitis in C57BL/6 mice receiving dextran sodium sulfate (DSS) in their drinking water, using additional treatment of DSS-exposed mice with combined human Igs, commercial IVIG (given IM, hence hereafter IMIG) as a source of pooled anti-idiotype Ig, and human anti-Tet as immune Ig.

### Example 6

Autoimmunity Experiment Protocol with Polyclonal Human IMIG and Polyclonal Human Anti-Tetanus Ig

Wild-type (WT) C57BL/6 female mice, with high susceptibility to DSS-induced colitis, were used throughout and purchased from the Jackson laboratories (Bar Harbor, Me.). All mice were housed five per cage under specific pathogen-free conditions, allowed standard diet and water ad libitum, and used at 6-8 wk of age.

Animal experimentation was performed following guidelines of an accredited animal care committee (CACC) at Cedarlane Laboratories. Humane endpoints were used in all studies, with mice monitored daily. Animals were euthanized (overdose with pentobarbital) when they were exhibiting signs of distress (weight loss≥25%; hunched posture; diarrhea; loss of active movements). Mortality was seen only in studies of acute colitis, with mean (averaged over ˜100 mice) ≤15%. Animals with diarrhea (but weight loss <25%) received daily ip injections of saline (1 ml×3 at 8 hr intervals) to avoid dehydration [1].

Acute DSS colitis was induced by giving mice distilled drinking water containing 3% (wt./vol) DSS (Mol.Wt.=40 kDa; ICN Biochemicals, Aurora, Ohio) for 7 days [8,11]. For induction of chronic colitis, mice were treated with 5 days of 2.5% DSS followed by 7 days of normal drinking water for a total of 3 cycles. Body weight was measured three times/week throughout the experiment. The maximum mortality observed following induction of acute colitis was 15% averaged over all studies, while no significant mortality was seen any groups in the chronic colitis model. Maximum weight loss in either model was ˜25%. All mice suffering from acute colitis recovered weight loss within 7-10 days post cessation of DSS exposure [1].

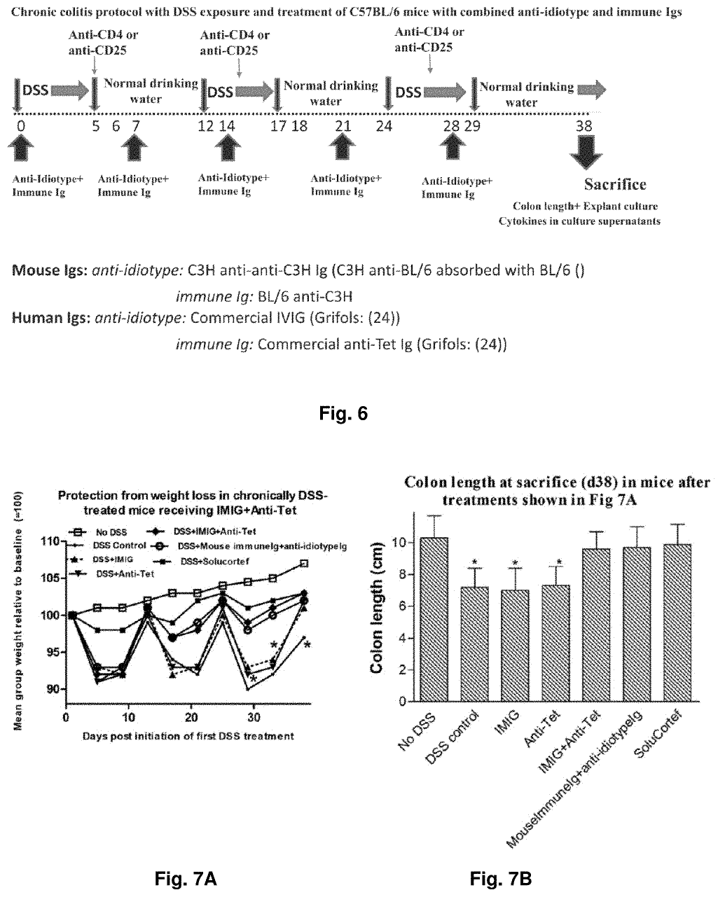

In some groups, mice receiving DSS were also treated with combinations of homologous (mouse) or heterologous (human) anti-idiotype Ig and immune Ig, given intramuscularly at separate sites at weekly intervals for a maximum of 5 injections, beginning on the day of initial exposure to DSS. The mouse Igs used were 75 μg of pooled polyclonal anti-idiotype Ig (C3H anti-anti-C3H Ig (C3H anti-BL/6 absorbed with BL/6)) and 10 μg pooled polyclonal BL/6 anti-C3H immune Ig [7]. The human Igs used were 75 μg of human commercial IVIG (pooled anti-idiotype [12-15], given intramuscularly, hence IMIG) or 10 μg human anti-Tet Ig (both from GRIFOLS, Canada [9,10]). The protocols used for DSS and Ig treatment are shown schematically in FIG. 11 (acute colitis) and FIG. 6 (chronic colitis) respectively. A control DSS treated group in each acute/chronic colitis model received Solu-Cortef (6 μg/mouse on alternate days) throughout the course of study as an alternate way of suppressing the disease.

For the protocol shown in FIG. 11, both homologous (mouse) and heterologous (human) Igs were used. Data for such studies are shown in FIGS. 12A, 12B, 12C.

Where CD4⁺T cells were depleted from mice with chronic DSS-induced colitis, mice received 50 μg/mouse of anti-mouse CD4 mAb (GK1.5), purchased from Cedarlane Labs, Hornby, Ontario, iv three times (at completion of the first DSS treatment and subsequently midway through subsequent DSS treatments as shown in FIG. 6). All animals were sacrificed by cervical dislocation under halothane anaesthesia.

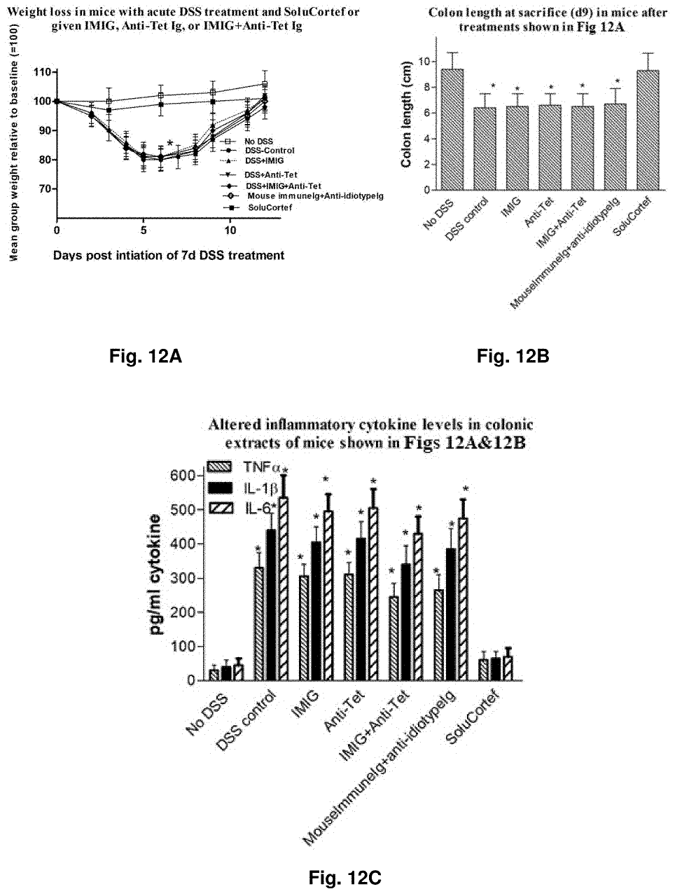

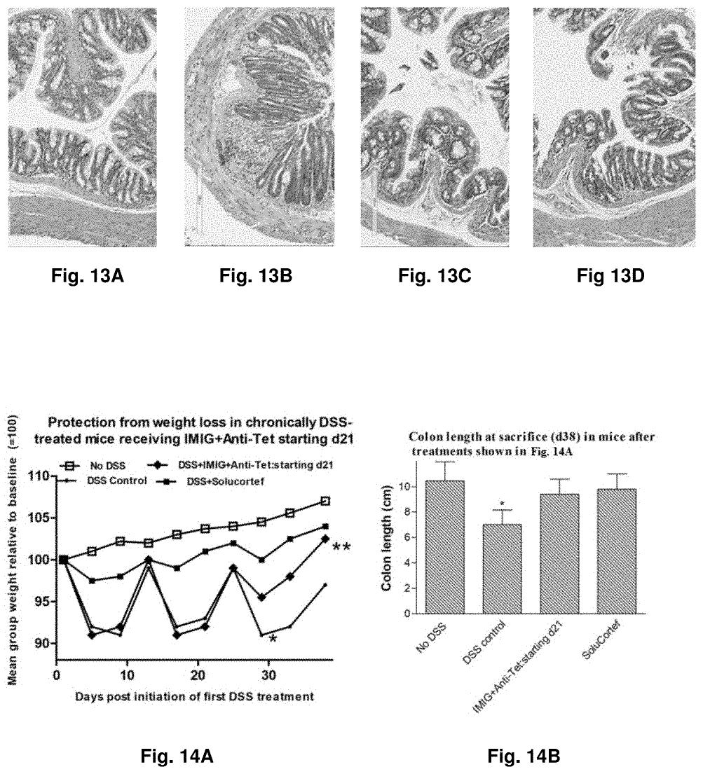

Colonic tissue was harvested from mice at sacrifice after 3 rounds of DSS treatment for induction of chronic colitis and colon lengths measured [1]. 0.5 cm fragments of the colon were fixed in 10% neutral-buffered formalin for 24 h and paraffin cross-sections (5 μm) were prepared and stained with hematoxylin and eosin (H&E). Representative H&E staining of colon segments are shown in FIGS. 13A, 13B, 13C, and 13D.

The colons were washed three times with cold PBS containing 100 IU penicillin and 100 μg/ml streptomycin and opened longitudinally. Colon strips 3 mm in length were cultured in 500 μl of supplemented RPMI 1640 culture medium at 37° C. with 5% CO2 humidified air for 24 h, and supernatants were collected after particulate material removal by centrifugation for 10 min at 1000×g. Supernatants were assayed in ELISA using commercial capture/detection antibodies (BioLegend™) for IL-1 (3, IL-4, IL-6, IL-10, IL-12, IL17A, IFNγ, TNFα and TGFβ. Capture antibodies were coated on one Elisarray microplate and 50 μl of samples were added to the wells of the plate. After 2 h incubation and exhaustive washing to remove unbound proteins, 100 μl of biotinylated detection antibodies were added. Thereafter, an avidin-horseradish peroxidase conjugate was added after 1 h incubation. After further washing substrate solution was added with the absorbance read at 450 nm. Manufacturer's cytokine standards were used for quantitation.

Small strips of colon isolated as above from pools of 2-3 mice/group were incubated in 15 ml of pre-digestion PBS solution with 5 mM EDTA and 20 mM HEPES (pH=7.2) for 15 minutes at room temperature with constant stirring with a magnetic bar (˜50 rpm/min). Strips were harvested, resuspended in more of the same fresh medium, and the process repeated ×3. After the final wash, samples were in 20 ml of digestion medium (alpha MEM with 20% FCS containing 2 mg/ml collagenase Type3 and 0.2 mg/ml DNase I (both from Sigma biochemical, Mississauga, Ontario, Canada)) at 37° C. for 60 min, again stirring at 50 rpm. Supernatant containing LP cells was retained in a 50 ml tube, and the digestion process repeated ×2 with fresh digestion medium on each occasion. Combined supernatants were strained through a 70 um cell strainer, centrifuged for 5 mins at 1300 rpm at 4° C., and resuspended in 5 ml PBS containing 0.5% BSA and 2mMEDTA for counting and subsequent FACS analysis.

CD4-PE-Cy7, CD3-FITC and Foxp3-PE (BioLegend, San Diego, USA) were used to identify different cell populations in LP on a BD LSR flow cytometer. Cell surface staining and intracellular staining were conducted according to the manufacturer's instructions.

Statistical significance used the Student t test or one-way ANOVA followed by Tukey tests. P-values less than 0.05 were considered statistically significant and shown in the figures. In studies comparing weight loss in different groups of DSS-treated mice receiving different immunomodulatory regimens, curves were compared using Mann-Whitney U-tests.

Results

1. Combined Immune Ig and Anti-Idiotype Ig does not Ameliorate Body Weight Loss, and Inflammatory Cytokine Production in DSS-Induced Acute Colitis

The inventors previously reported that in mice pre-treated (beginning 14 d before DSS exposure) to combinations of autologous (mouse) immune Ig and anti-idiotype Ig, a significant attenuation of weight loss induced by acute DSS exposure was seen, along with diminished shortening of colon length and decreased inflammatory cytokine production [7]. Given that this protocol was far removed from a clinically applicable therapy, the inventors first explored whether a similar attenuation of markers of acute inflammation was seen in mice receiving acute (7 d) DSS exposure, but given the combined Igs only from the time of DSS treatment, not as pre-exposure to DSS as shown in FIG. 11. Since the inventors also previously found that heterologous (human) Igs are as effective in immune modulation in an allergic inflammation model as mouse Igs [9,10], some groups in this study received human anti-Tet or IMIG, alone or in combination, along with a separate group receiving combined immune and anti-idiotype mouse Igs. As a “gold standard” for suppression of colitis a separate group of mice received Solu-Cortef given intramuscularly on alternate days. Data combined from two such studies are shown in FIGS. 12A, 12B, and 12C. In FIG. 12A, changes in body weight are expressed as a percent weight loss from baseline and plotted versus time post initiation of DSS treatment (x-axis). Data points represent mean±SD for each group. In FIGS. 12B and 12C, changes in colon length and 24 hr cytokine production from colon explant cultures respectively were assessed on day 9 following initiation of DSS treatment for mice shown in FIG. 12A and are expressed as mean±SD. All data points are pooled from 2 independent experiments with a total of 10 mice/group. *p<0.05 compared with no DSS exposure, or DSS+Solu-Cortef.

It is apparent from these data that while Solu-Cortef protected mice from multiple measures of pathology associated with acute colitis in this model, no groups receiving immune Igs, or anti-idiotype Igs, alone or in combination, of either mouse or human origin, was protected from pathology, as assessed by weight loss, colon length or production of inflammatory cytokines (see * in FIGS. 12A, 12B, and 12C in comparison to control untreated control mice or Solu-Cortef treated mice). This is in marked contrast to previous findings where combined Igs given (at two weekly doses) before DSS exposure led to attenuation of inflammation [7]. The inventors speculate that in the acute onset colitis model described in FIGS. 11, 12A, 12B, and 12C, the lack of time between immunomodulation and DSS exposure and induction of acute colitis, prevented the attenuation of disease described previously [7]. Accordingly in all subsequent studies described below the inventors have focused only on modifying induction of inflammation in a chronic colitis model (see FIG. 6 for schematic), where disease is less severe and occurs over a more prolonged time (remitting/relapsing).

2. Combined Immune Ig and Anti-Idiotype Ig Reduced Weight Loss and Shortened Colon Length in DSS-Induced Chronic Colitis

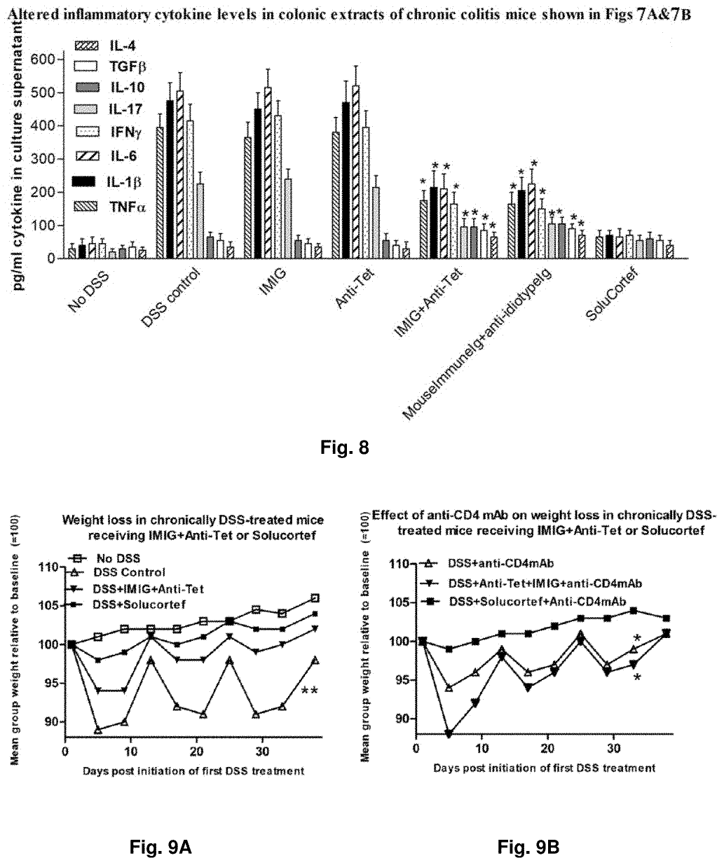

DSS-induction of chronic colitis followed the protocol shown in FIG. 6. Mice received 5 days of DSS exposure followed by 7 days of normal drinking water. Ig treatments were given at the start of the first cycle with DSS and weekly thereafter for a total of 5 injections. Body weight changes in groups of mice are shown in FIG. 7A, along with data for control (non-DSS treated) mice and DSS treated mice receiving Solu-Cortef.

In FIG. 7A the changes in body weight are expressed as a percent weight loss from baseline (y-axis). Data points in FIG. 7B represent mean±SD for a total of 10 mice/group. SD (<10% of mean) are not shown in FIG. 7A to retain clarity in the figure. In FIG. 7B, changes in colon length were assessed on day 38 following 3 cycles of DSS treatment for the groups of mice shown in FIG. 7A (see FIG. 6). All data points are pooled from 2 independent experiments with a total of 10 mice/group. *p<0.05 compared with no DSS exposure, or DSS+Solu-Cortef (Mann-Whitney test for FIG. 7A; ANOVA for FIG. 7B).

Representative H&E staining of colonic tissue from different groups of mice are shown in FIGS. 13A, 13B, 13C, and 13D.

In all DSS treated groups except those receiving Solu-Cortef a loss of weight occurred in the first cycle of DSS treatment (by ˜day 5). All mice recovered during the rest period when drinking plain water. Thereafter, in mice receiving combined Ig treatments, as well as in Solu-Cortef treated mice, attenuation of weight loss was seen in the second and third cycle of DSS treatment (see days 18-20 and 29-31 respectively). No such protection was seen in mice receiving only anti-Tet Ig alone, or anti-idiotype (IMIG) alone. The attenuation of weight loss was seen in groups receiving both homologous (mouse) and heterologous (human) Ig treatment. By day 38 weight was regained in all groups of DSS treated mice. [Note that the inventors have also seen a similar protection from weight loss in DSS-treated mice receiving combined IMIG and Anti-Tet, when immunomodulation was initiated only after two complete cycles of induction of colitis in the model described (i.e. IMIG and anti-Tet Ig given only on days 17, 24 and 31 post initiation of the first cycle of DSS treatment as per FIG. 6). Data for one such study of mice again sacrificed at day 38 (per FIG. 6) are shown in FIGS. 14A and 14B].

In FIG. 14A, changes in body weight are again expressed as a percent weight loss from baseline (y-axis). Data points in FIG. 14B represent mean±SD for a total of 10 mice/group. SD (<10% of mean) are not shown in FIG. 14A to retain clarity in the figure. In FIG. 14B, changes in colon length were assessed on day 38 following 3 cycles of DSS treatment for the groups of mice shown in FIG. 14A (see FIG. 6). *p<0.05 compared with no DSS exposure, or DSS+Solu-Cortef; **p<0.05 compared with DSS control (Mann-Whitney test for FIG. 14A; ANOVA for FIG. 14B).

As shown in FIGS. 7B, 14A and 14B, similar significant differences were seen in the change of colon length among the groups in the chronic colitis model compared with control mice (no DSS or DSS+Solu-Cortef). The average colon length in DSS treated mice was reduced by ˜13 mm at the termination of the study in FIG. 7B, except in DSS treated mice receiving also combined Ig treatments (mouse or human origin) or DSS+Solu-Cortef.

3. Combined Immune Ig and Anti-Idiotype Ig Inhibits Inflammatory Cytokine Release in Chronic Colitis:

FIG. 8 shows cytokine expression analysis in 24 hr culture supernatants of colonic explants from mice. The groups of mice are shown in FIGS. 7A and 7B. 24 hr colonic explant cultures harvested at 38 d (per FIG. 6) were assayed for different inflammatory cytokines using commercial ELISAs (BioLegend: USA). Data show mean (+SD) of triplicate measurements from 3 cultures/group. *, indicates p<0.05 compared with DSS treated groups receiving no additional treatment, or IMIG or anti-TetIg alone (ANOVA).

As reported previously [1], DSS treated mice showed increased expression levels of inflammatory cytokines, IFNγ, TNFα, IL-1β, IL-17 and IL-6, and levels remained high even after treatment with anti-TetIg or IMIG alone. The inventors and others have provided data to suggest that infiltrating CD3+CD4+ cells are a significant source of inflammatory cytokines in chronic colitis [1,16]. No such increased inflammatory cytokine release was seen in mice receiving DSS and Solu-Cortef. Interestingly, mice receiving combined immune Ig and anti-idiotype Ig, again of either mouse or human origin, also showed attenuation of release of these inflammatory cytokines. In these latter two groups of mice there was also a significantly increased production of anti-inflammatory cytokines (IL-10, TGFβ) compared with other groups, and a trend to augmented IL-4 release also. This is consistent with the inventors' previous report which indicated that immunomodulation of chronic colitis was associated with a polarization towards type-2 cytokine production in such mice [1,9,10].

4. Increased CD3+Foxp3+ Tregs in PP of DSS Chronic Colitis Mice Receiving Combined Immune Ig and Anti-Idiotype Ig

As noted above, earlier studies also showed the infiltrating mononuclear cells in chronic colitis were enriched for CD3+CD4+T cells [1,16]. It has been reported that there was a relative increase in the Foxp3:CD3 ratio in histological sections of mice with chronic colitis receiving immunomodulatory anti-inflammatory treatment, along with an increase in expression of the chemokine receptor CCR4, an attractant to Treg cells, and of the CCR4 ligands (CCL-17 and CCL-22) [1,17]. Using FACS analysis of PP cells isolated from colon tissue of individual mice in the different groups shown in FIGS. 7A and 7B, the inventors observed (see Table 1 below) a similar increase in the numbers of Foxp3+CD3+ cells, along with an increased Foxp3:CD3 ratio, in the group of DSS treated mice receiving combined Ig treatment. Importantly the numerical CD3+ infiltrate was most pronounced in mice receiving DSS but without Solu-Cortef or combined Ig treatment, the groups showing the most pronounced disease and the greatest inflammatory (CD3+CD4+) infiltrate (see FIGS. 7A, 7B, 8, also [1,16]).

TABLE 1
FACS staining of CD3⁺ and Foxp3⁺ cells in PP cells isolated from colonic tissue of mice shown in FIG. 7A.

| | Treatment of mice as per FIG. 7A | | | | | | |
|---|---|---|---|---|---|---|---|
| | | | | | DSS + | DSS + | |
| | | | | | Anti-Tet + | MouseIg + | DSS + |
| Cell staining | Control | DSS alone | DSS + And-TetIg | DSS + IMIG | IMIG | and-idiotype | Solucortef |
| % Ant-CD3 | 3.1 ± 2.0 | 20 ± 4.5* | 19 ± 3.3* | 22 ± 4.7* | 7.4 ± 3.8** | 8.3 ± 3.1** | 4.4 ± 2.2 |
| % Anti-Foxp3 | 0.1 ± 0.1 | 0.4 ± 0.3* | 0.4 ± 0.2* | 0.4 ± 0.2* | 0.6 ± 0.2* | 0.6 ± 0.3* | 0.2 ± 0.2 |
| Foxp3:CD3 | 0.03 | 0.02 | 0.02 | 0.02 | 0.08* | 0.07* | 0.04 |

Peyer's Patch cells were harvested from 2-3 mice/group and stained as described in the Material and Methods.
*P < 0.05 compared with no DSS or DSS + Solucortef;
**p < 0.05 compared with DDS + anti-Tet or IMIG alone.

5. Effect of T Depletion on Protection from DSS-Induced Chronic Colitis

Previous data [1] and indeed FACS analysis of PP cells isolated from mice in FIG. 7A, 7B, 8, suggest that in untreated chronic colitis a significant contribution to inflammatory pathology is a result of infiltrating CD4+ T cells, while following immunomodulation (in the case above after infusion of immune Ig and anti-idiotype Ig), infiltrating Foxp3+ Tregs contribute to amelioration of disease. This is consistent with data in an OVA-allergy model in mice suggesting that protection from allergic inflammation following treatment with immune Ig+anti-idiotype Ig was associated with increased Tregs [9,10]. To explore this further the inventors performed the following study.

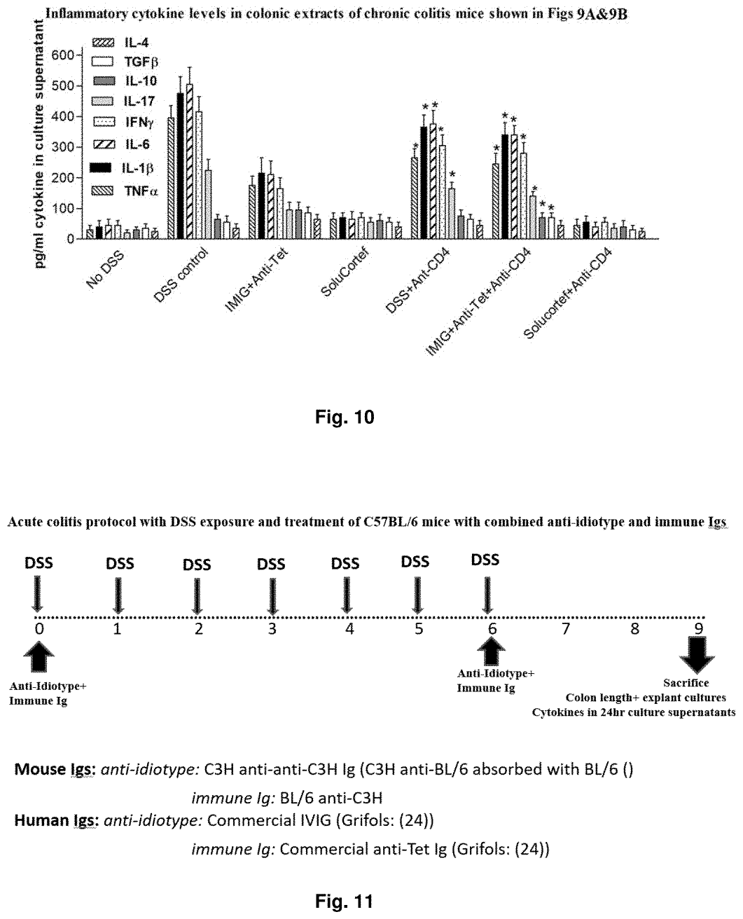

Groups of mice receiving DSS exposure for chronic colitis induction (per FIG. 6) received in addition immunomodulation with combined Ig treatment with human anti-Tet-IMIG as before, or Solu-Cortef as a “gold standard”. Subgroups of similarly treated animals also received iv infusions of 50 μg/mouse of anti-CD4 mAb (GK1.5) given on 3 occasions at the times shown in FIG. 6. Body weights were monitored as before, and at conclusion of the study colon explant cultures were used to assess cytokine production by ELISA, as in FIG. 8. Data for this study are shown in FIGS. 9A, 9B, 10 (5 mice/group).

Referring to FIGS. 9A and 9B, 5 mice/group received treatment as shown in FIG. 6, 7A, 7B, but in addition anti-CD4 mAb was given on 3 occasions to groups in FIG. 9B, as noted in FIG. 6. Group mean changes in body weight are shown, but SD (<10% of mean) are again not shown to retain clarity in the figure. All data represent mean±SD of five mice. **p<0.05 ** compared with all other groups in FIG. 9A; *p<0.05 compared with same group in FIG. 9A not receiving anti-CD4 mAb.

Referring to FIG. 10, all data points represent mean±SD in triplicate cultures with cells pooled from five mice/group. **p<0.05 ** compared with all other groups in FIG. 9A; *p<0.05 compared with same group in FIG. 9A not receiving anti-CD4 mAb.

As shown in FIG. 9A, both Solu-Cortef and combined anti-Tet+IMIG protected mice from weight loss after induction of chronic colitis (see also FIG. 7A). Once again this was associated with attenuation of release of inflammatory cytokines from colon explant cultures (see second and third groups of histograms in FIG. 10 and compare again with FIG. 8). Importantly, for mice receiving DSS treatment only, release of inflammatory cytokines was attenuated, and body weight loss diminished, in mice receiving anti-CD4 treatment (FIG. 9B, 10), consistent with a major contribution of CD4+ T cells to the pathology in these mice. Equally interesting however, in mice with immunomodulation by anti-Tet+IMIG, but not Solu-Cortef, infusion of anti-CD4 mAb worsened weight loss, and led to augmented release of inflammatory cytokines from colon cultures (FIG. 9B, 10). Again, this is consistent with the hypothesis that attenuation of disease following combined Ig treatment is a function of augmented activity in Tregs as reported elsewhere [9,10] and thus inhibition (or removal) of these cells suppressed the protective effect seen.

Discussion

Loss of an intact mucosal barrier and altered regulation of immunity to common luminal bacterial antigens precedes the development of IBD [2-6]. DSS is directly toxic to the colonic epithelial cells of the basal crypts, breaking down the mucosal epithelial barrier, with subsequent entry of luminal microorganisms into the mucosa to trigger an inflammatory response is triggered [11,18]. Supporting this hypothesis that dysregulation of the inflammatory response controls the pathogenesis of IBD are data showing a role for Toll-like receptors (TLRs) in disease [19]; of NIrp3, and the “inflammasome” in DSS-induced colitis [20,21]; and altered release of inflammatory cytokines in disease [22-26]. It should be noted however, as stressed in an earlier publication [1], that while the DSS-induced colitis model has been widely used by investigators exploring factors implicated in human disease [27], there are many variables which influence comparison of data from murine DSS studies to humans [28], and even different murine DSS studies with one another, including the absence/presence of different TLRs in mice/man [29-33].

It has been reported previously on a DSS colitis model in different sub-strains of C57BL/6 mice which showed an important role for attenuation of inflammatory cytokine production by myeloid cells in reversal of pathology in acute colitis, while regulatory Foxp3+ Treg levels seemed more critical to control CD4+ T cell mediated inflammation in chronic colitis [1,33,34]. Multiple other cells, including, but not limited to, B cells [35] and Bregs [36], innate lymphoid cells [37] and macrophage/dendritic cells [38-40]. The current study has used an alternative strategy to regulate colitis in wild-type C57BL/6 mice, known to be susceptible to DSS colitis, using a combination of immune and anti-idiotype Igs which the inventors have shown is capable of altering inflammation in multiple models of disease in experimental animals, including transplant rejection, inflammatory breast cancer, acute colitis and OVA-induced allergic immunity [7,9,10]. Moreover, and further extending the applicability of this model across species, including man, the inventors have again assessed whether Igs from different vertebrate species can act in the same fashion in mice [9,10]. The inventors showed first, that in apparent contradiction to an earlier report [7], the inventors were unable to influence acute colitis in C57BL/6 mice using combined human or even mouse Igs if treatment was initiated from the time of first exposure to DSS (FIG. 12A, 12B, 12C). In retrospect, since the inventors have argued that immunotherapy in this model likely involves enhanced numbers/activity in Tregs [9,10], it is likely that simultaneous DSS and Ig delivery led to the failure of immunotherapy, since there was no time to induce a regulatory (protective) T cell population. The success in the earlier study [7] was likely attributable to the pre-treatment of mice with immune Ig+ ant-idiotype Ig (for 14 d) before DSS exposure in that study. In support of this explanation, the inventors note that even in the chronic colitis model studied above, where diseases was less severe and of a relapsing/remitting nature, in mice receiving DSS and combined IMIG and immune Ig from initiation of DSS (FIG. 6, 7A, 7B, 8, 9A, 9B, 10), during the first cycle of DSS exposure there was still little benefit from combined immunotherapy with immune Ig+ anti-idiotype Ig. In contrast, Solu-Cortef was active in suppressing disease at all times (FIG. 7A, 7B, 8, 9A, 9B, 10).

Most importantly however, the data do indicate that there is a therapeutic role for the combined Ig treatment discussed which is evident within 2-3 weeks from the first DSS exposure and is maintained thereafter (FIG. 7A, 7B, 8, 9A, 9B, 10). Moreover, even when treatment with IMIG and anti-Tet was delayed to the end of a second cycle of DSS treatment, i.e. with colitis already established, the combination of IMIG and anti-Tet did lead to significant attenuation of colitis (see FIG. 14A, 14B).

Attenuation of colitis in the model shown in FIG. 6, as previously described in an OVA-allergy model, was associated with attenuation of inflammatory cytokine production (FIG. 8), and an augmented presence/activity in Tregs in colonic tissue (Table 1; FIG. 9A, 9B, 10). While anti-CD4+mAb treatment led to some suppression of colitis/inflammatory cytokine release in mice not receiving combined Ig treatment (FIG. 9A, 9B, 10), as predicted given the important role of CD4+ cells in chronic DSS colitis (ref [1] and above), in mice receiving combined Ig treatment the loss of CD4+ (Tregs) actually led to attenuation of protection afforded by immune Ig+ anti-idiotype (see also [9,10]). It is important to note however, that the protection afforded by combined Ig treatment in mice was seen regardless of the source of Ig used, including using both homologous (mouse) and heterologous (commercial human) Igs (anti-Tet and IMIG).

The trend over the past decade in treatment of refractory inflammatory bowel disease in man has moved from use of nonspecific therapy (immunosuppressive drugs/Steroids etc.) to the use of so-called “Biologics” (or biotechnological) drugs. The latter are moieties that are produced by biological systems and target molecules or pathways which prior research has shown to be involved in the inflammatory cascade that is triggered during IBD. Anti-TNF blockers (e.g., infliximab, adalimumab, certolizumab pegol, and golimumab) have been at the forefront of this therapy [41]. However, given that a finite number of individuals fail to respond, the side effects associated with the drug, and a described loss of efficacy with time, there has been a move to develop other reagents, foremost amongst which are mAbs to other cytokines [42,43] or molecules regulating cell trafficking [44], and oral small molecule inhibitors [45]. Common to all of these treatments is the concern regarding acute reactions (hypersensitivity etc.) and delayed effects of long-term administration (infection, e.g. TB, and drug toxicity/malignancy). In this regard the regimen used above, using pooled human anti-Tet Ig and IVIG, may offer a unique and safe alternative to these regimens, given that the therapy aims to re-dress an imbalance in the host immune system rather than supplant host defence with an exogenous source. It is intriguing to speculate that the therapy may be best used as an adjunct treatment after initial more conventional therapy gains early “control” of disease, particularly since the inventors have shown that the combined Ig treatment used takes some time to develop effective control of inflammation (see FIG. 12A, 12B, 12C and FIG. 7A, 7B, 8), but can be effective even in the face of previously established disease (FIG. 14A, 14B).

### CONCLUSION

The inventors have shown that in a model of chronic inflammatory colitis, combination therapy using polyclonal immune Ig and anti-idiotype Ig can protect from disease in mice. This protection develops within 14 d of onset of colitis, and is evident by attenuation of weight loss, shortened colon length, and release of inflammatory cytokines. It is seen whether the combined Igs used are of homologous (mouse) or heterologous (human) species origin and depends upon development of a suppressive CD4+ cell population. These data are similar to those reported in a previously used model showing protection from allergic disease in mammals, and this therapy may have a widespread clinical utility.

In the foregoing description, exemplary modes for carrying out the invention in terms of examples have been described. However, the scope of the claims should not be limited by those examples, but should be given the broadest interpretation consistent with the description as a whole. The specification and drawings are, accordingly, to be regarded in an illustrative rather than a restrictive sense.

### REFERENCES

1. Chen Z, Yu K, Zhu F, Gorczynski R M. Over-expression of CD200 protects mice from dextran sodium sulfate induced colitis PlosOne, 2016 Feb. 3; 11(2): e0146681. doi: 10.1371/journal.pone.0146681.
2. Strober W, Fuss I, Mannon P. The fundamental basis of inflammatory bowel disease. J Clin Invest. 2007; 117: 514-521.
3. Cho J H. The genetics and immunopathogenesis of inflammatory bowel disease. Nat Rev Immunol. 2008; 8: 458-466.
4. Xavier R J, Podolsky D K (2007) Unravelling the pathogenesis of inflammatory bowel disease. Nature. 2007; 448: 427-434.
5. Khor B, Gardet A, Xavier, R J. Genetics and pathogenesis of inflammatory bowel disease. Nature. 2011; 474: 3017-317
6. Strober W, Fuss I J. Proinflammatory cytokines in the pathogenesis of inflammatory bowel diseases. Gastroenterology. 2011; 140: 1756-1767.
7. Gorczynski R M and Hoffmann G W. Towards a new kind of vaccine: a logical extension of the symmetrical immune network theory Interact J Med Res. 2017: PMID: 28879488 DOI: 10 2196/ijmr.7612
8. Wirtz S, Neufert C, Weigmann B, Neurath M F. Chemically induced mouse models of intestinal inflammation. Nat Protoc. 2007; 2: 541-546.
9. Gorczynski, R. M., Maqbool, T. & Hoffmann, G. Mechanism(s) of prolonged attenuation of allergic responses after modulation of idiotypic regulatory network. Allergy Asthma Clin Immunol 15, 79 (2019) doi:10.1186/513223-019-0393-7
10. Gorczynski, R M and Hoffmann G W: A novel mechanism for induction of

Tregs to control allergic disease. Int Med Rev. 2019:DOI: http://dx.doi.org/10.18103/imr.v5i8.839
11. Okayasu I, Hatakeyama S, Yamada M, Ohkusa T, Inagaki Y, Nakaya R. A novel method in the induction of reliable experimental acute and chronic ulcerative colitis in mice. Gastroenterology. 1990; 98: 694-702.
12. Schaub A, von Gunten S, Vogel M, Wymann S, Ruegsegger M, Stadler B M, Spycher M, Simon H U, Miescher S. Dimeric IVIG contains natural anti-Siglec-9 autoantibodies and their anti-idiotypes. Allergy. 2011; 66:1030-7.
13. DeKeyser F, DeKeyser H, Kazatchkine M D, Rossi F, Dang H, Talal N. Pooled human immunoglobulins contain anti-idiotypes with reactivity against the SLE-associated 4B4 cross-reactive idiotype. Clin Exp Rheumatol. 1996; 14:587-91.
14. Caccavo D, Vaccaro F, Ferri G M, Amoroso A, Bonomo L. Anti-idiotypes against antiphospholipid antibodies are present in normal polyspecific immunoglobulins for therapeutic use. J Autoimmun. 1994; 7:537-48.
15. Dietrich G, Kaveri S V, Kazatchkine M D. Modulation of autoimmunity by intravenous immune globulin through interaction with the function of the immune/idiotypic network. Clin Immunol Immunopathol. 1992 January; 62(1 Pt 2): S73-81. Review.
16. Takedatsu H, Michelsen K S, Wei B, Landers C J, Thomas L S, Dhall D et al. TL1A (TNFSF15) regulates the development of chronic colitis by modulating both T-helper 1 and T-helper 17 activation. Gastroenterology. 2008; 135: 552-567.
17. Yu K, Chen Z, Khatri I, Gorczynski R M. CCR4 dependent migration of Foxp3+ Treg cells to skin grafts and draining lymph nodes is implicated in enhanced graft survival in CD200tg recipients. Immunol Lett. 2011; 141: 116-122.
18. Perse M, Cerar A. Dextran sodium sulphate colitis mouse model: traps and tricks. J Biomed Biotechnol 2012; 2012: 718617.
19. Uematsu S, Jang M H, Chevrier N, Guo Z, Kumagai Y, Yamamoto M et al. Detection of pathogenic intestinal bacteria by Toll-like receptor 5 on intestinal CD11c+ lamina propria cells. Nat Immunol. 2006; 7: 868-874.
20. Zaki M H, Boyd K L, Vogel P, Kastan M B, Lamkanfi M, Kanneganti T D. The NLRP3 inflammasome protects against loss of epithelial integrity and mortality during experimental colitis. Immunity. 2010; 32: 379-391.
21. Bauer C, Duewell P, Mayer C, Lehr H A, Fitzgerald K A, Dauer M et al. Colitis induced in mice with dextran sulfate sodium (DSS) is mediated by the NLRP3 inflammasome. Gut. 2010; 59: 1192-1199.
22. Alex P, Zachos N C, Nguyen T, Gonzales L, Chen T E, Conklin L S et al. Distinct cytokine patterns identified from multiplex profiles of murine DSS and TNBS-induced colitis. Inflamm Bowel Dis. 2009; 15: 341-352.
23. Pizarro T T, Cominelli F. Cytokine therapy for Crohn's disease: advances in translational research. Annu Rev Med. 2007; 58: 433-444.
24. Di Sabatino A, Biancheri P, Rovedatti L, MacDonald T T, Corazza G R. New pathogenic paradigms in inflammatory bowel disease. Inflamm Bowel Dis. 2012; 18: 368-371.
25. Kim Y S, Lee M H, Ju A S, Rhee K J. Th17 responses are not induced in dextran sodium sulfate model of acute colitis. Immune Netw. 2011; 11: 416-419.
26. Rovedatti L, Kudo T, Biancheri P, Sarra M, Knowles C H, Rampton D S et al. Differential regulation of interleukin 17 and interferon gamma production in inflammatory bowel disease. Gut. 2009; 58: 1629-1636.
27. Melgar S, Karlsson L, Rehnstrom E, Karlsson A, Utkovic H, Jansson L et al. Validation of murine dextran sulfate sodium-induced colitis using four therapeutic agents for human inflammatory bowel disease. Int Immunopharmacol. 2008; 8: 836-844.
28. Rakoff-Nahoum S, Paglino J, Eslami-Varzaneh F, Edberg S, Medzhitov R. Recognition of commensal micromicrobiota by toll-like receptors is required for intestinal homeostasis. Cell. 2004; 118: 229-241.
29. Heimesaat M M, Fischer A, Siegmund B, Kupz A, Niebergall J, Fuchs D et al. Shift towards pro-inflammatory intestinal bacteria aggravates acute murine colitis via Toll-like receptors 2 and 4. PLoS One. 2007; 2: e662.
30. Fukata M, Michelsen K S, Eri R, Thomas L S, Hu B, Lukasek K et al. Toll-like receptor-4 is required for intestinal response to epithelial injury and limiting bacterial translocation in a murine model of acute colitis. Am J Physiol Gastrointest Liver Physiol. 2005; 288: G1055-1065.
31. Araki A, Kanai T, Ishikura T, Makita S, Uraushihara K, Iiyama R et al. MyD88-deficient mice develop severe intestinal inflammation in dextran sodium sulfate colitis. J Gastroenterol. 2005; 40: 16-23.
32. Szebeni B, Veres G, Dersofi A, Rusai K, Vannay A, Mraz M et al. Increased expression of Toll-like receptor (TLR) 2 and TLR4 in the colonic mucosa of children with inflammatory bowel disease. Clin Exp Immunol. 2008; 151: 34-41.
33. Fantini M C, Becker C, Tubbe I, Nikolaev A, Lehr H A, Galle P et al. Transforming growth factor beta induced FoxP3+ regulatory T cells suppress Th1 mediated experimental colitis. Gut. 2006; 55: 671-680.
34. Eastaff-Leung N, Mabarrack N, Barbour A, Cummins A, Barry S. Foxp3+ regulatory T cells, Th17 effector cells, and cytokine environment in inflammatory bowel disease. J Clin Immunol. 2010; 30: 80-89.
35. Stevceva L, Pavli P, Husband A J, Doe W F. The inflammatory infiltrate in the acute stage of the dextran sulphate sodium induced colitis: B cell response differs depending on the percentage of DSS used to induce it. BMC Clin Pathol. 2001; 1: 3.
36. Mizoguchi E, Mizoguchi A, Preffer F I, Bhan A K. Regulatory role of mature B cells in a murine model of inflammatory bowel disease Int. Immunol. 2000; 12: 597-605.
37. Walker J A, Barlow J L, McKenzie A N. Innate lymphoid cells—how did we miss them? Nat Rev Immunol. 2013; 13: 75-87.
38. Rivollier A, He J, Kole A, Valatas V, and Kelsall B L. Inflammation switches the differentiation program of Ly6Chi monocytes from antiinflammatory macrophages to inflammatory dendritic cells in the colon J Exp Med. 2012; 209: 139-155
39. Geem, D., Medina-Contreras, O., Kim, W., Huang, C. S., Denning, T. L. Isolation and Characterization of Dendritic Cells and Macrophages from the Mouse Intestine. J. Vis. Exp. (63), e4040, doi:10.3791/4040 (2012).
40. Bradford E. Berndt B E, Zhang M, Chen G-H, Huffnagle G B, Kao J Y The Role of Dendritic Cells in the Development of Acute Dextran Sulfate Sodium Colitis J Immunol 2007 179:6255-6262
41. Pagnini C, Pizarro T T, and Cominelli F. Novel Pharmacological Therapy in Inflammatory Bowel Diseases: Beyond Anti-Tumor Necrosis Factor Front. Pharmacol. 2019: https://doi.org/10.3389/fphar.2019.00671
42. Feagan, B. G., Sandborn, W. J., Gasink, C., Jacobstein, D., Lang, Y., Friedman, J. R., et al. (2016). Ustekinumab as induction and maintenance therapy for Crohn's disease. N. Engl. J. Med. 375, 1946-1960. doi: 10.1056/NEJMoa1602773
43. Danese, S., Vermeire, S., Hellstern, P., Panaccione, R., Rogler, G., Fraser, G., et al. (2019). Randomised trial and open-label extension study of an anti-interleukin-6 antibody in Crohn's disease (ANDANTE I and II). Gut 68, 40-48. doi: 10.1136/gutjnl-2017-314562
44. Arseneau, K. O., Cominelli, F. (2015). Targeting leukocyte trafficking for the treatment of inflammatory bowel disease. Clin. Pharmacol. Ther. 97, 22-28.
45. Sandborn, W. J., Su, C., Sands, B. E., D'Haens, G. R., Vermeire, S., Schreiber, S., et al. (2017). Tofacitinib as induction and maintenance therapy for ulcerative colitis. N. Engl. J. Med. 376, 1723-1736. doi: 10.1056/NEJMoa1606910

1. The use of a combination of plasma immunoglobulin and antigen-specific immunoglobulin for the treatment or prevention of autoimmune disease.

2. The use of claim 1 wherein said autoimmune disease involves inflammation.

3. The use of claim 1 wherein said plasma immunoglobulin is homologous and said antigen-specific immunoglobulin is homologous.

4. The use of claim 1 wherein said plasma immunoglobulin is heterologous and said antigen-specific immunoglobulin is heterologous.

5. The use of claim 1 wherein said plasma immunoglobulin is pooled plasma immunoglobulin and wherein said antigen-specific immunoglobulin is anti-Tetanus toxoid Ig.

6. A pharmaceutical composition for the treatment or prevention of autoimmune disease, said pharmaceutical composition comprising:
plasma immunoglobulin; and
antigen-specific immunoglobulin.

7. The pharmaceutical composition of claim 6 wherein said autoimmune disease involves inflammation.

8. The pharmaceutical composition of claim 6 wherein said plasma immunoglobulin is homologous and said antigen-specific immunoglobulin is homologous.

9. The pharmaceutical composition of claim 6 wherein said plasma immunoglobulin is heterologous and said antigen-specific immunoglobulin is heterologous.

10. The pharmaceutical composition of claim 6 wherein said plasma immunoglobulin is pooled plasma immunoglobulin and wherein said antigen-specific immunoglobulin is anti-Tetanus toxoid Ig.

11. A method of treating or preventing autoimmune disease comprising administering injections of plasma immunoglobulin and antigen-specific immunoglobulin.

12. The method of claim 11 wherein said autoimmune disease involves inflammation.

13. The method of claim 11 wherein said plasma immunoglobulin is homologous and said antigen-specific immunoglobulin is homologous.

14. The method of claim 11 wherein said plasma immunoglobulin is heterologous and said antigen-specific immunoglobulin is heterologous.

15. The method of claim 11 wherein said plasma immunoglobulin is pooled plasma immunoglobulin and wherein said antigen-specific immunoglobulin is anti-Tetanus toxoid Ig.
